(19) United States
(12) Patent Application Publication
(10) Pub. No.: US 2015/0307841 A1
(43) Pub. Date: Oct. 29, 2015

(54) Composition for Promoting Differentiation and Proliferation of Neural Stem Cell and Neural Precursor Cell

(71) Applicant: University-Industry Cooperation Group Of Kyung Hee University, Gyeonggi-do (KR)

(72) Inventors: Yun Hee Kim Kwon, Seoul (KR); Dong Hyun Kim, Seoul (KR); Ma Eum Han, Seoul (KR)

(21) Appl. No.: 14/694,989

(22) Filed: Apr. 23, 2015

(30) Foreign Application Priority Data
Apr. 25, 2014 (KR) 10-2014-0049765
May 19, 2014 (KR) 10-2014-0059758

(51) Int. Cl. C12N 5/0793; A61K 31/704

(52) U.S. Cl. CPC C12N 5/0619; A61K 31/704; C12N 2501/999; C12N 2501/115; C12N 2506/08

(57) ABSTRACT

The present invention relates to a composition containing soyasaponin I for promoting differentiation or proliferation of neural stem cells or neural precursor cells. The composition may be usefully used as a composition for culturing cells in order to promote differentiation or proliferation of neural stem cells or neural precursor cells, and may be used to prevent or treat various neurological diseases caused by degenerative brain diseases and nerve damage by promoting differentiation and regeneration of neurons suitable for the transplantation sites.

### BACKGROUND OF THE INVENTION

1. Field of the Invention

The present invention relates to a composition for promoting differentiation or proliferation of neural stem cells or neural precursor cells containing soyasaponin I, a composition for culturing cells containing the same, and a pharmaceutical composition for preventing or treating neurological diseases containing the same.

2. Description of the Related Art

Neural stem cells (NSCs) are precursors to the neurons and glial cells that constitute the brain area during embryonic development. The neural stem cells divide to produce the same cells or the cells that will differentiate. The wrongly divided cells or unnecessary cells undergo apoptosis and the destiny of the surviving cells, i.e., their differentiation, is determined.

In normal animals, neural precursor cells proliferate and replace dead cells in some parts of the brain, including the hippocampus, even in adulthood (Identification of a neural stem cell in the adult mammalian central nervous system (1999), Cell 96, 25-34). Since a significant loss of neurons occurs in the parts affected by degenerative brain disease or brain damage, the proliferation of neural stem cells (neurogenesis) needs to be promoted. Because, for humans, the regeneration of the peripheral nervous system let alone the central nervous system is difficult, disabilities caused by degenerative brain diseases, industrial disasters, traffic accidents, and wars have become important social issues. Therefore, studies on the regeneration of the nervous systems have been drawing much attention.

Especially, in adults where neurogenesis is complete, damaged peripheral nerves undergo gradual Wallerian degeneration at the distal stumps toward nerve endings from the injured sites. For the central nervous system, axon regeneration hardly occurs due to various inhibitory substances such as the extracellular matrix proteins. Accordingly, a method for regenerating damaged central and peripheral nervous systems needs to be developed.

In Europe, transplantation of neural stem cells obtained from the fetus into patients with degenerative brain diseases, in particular, Parkinson's disease, has been clinically tried recently. After the transplantation, the patients exhibited very fast recovery and significant improvement in behaviors. However, since most of the transplanted cells die in 3 months to 1 year after the transplantation, the patients should receive the transplantation again. In addition, although a large number of patients need the transplantation, it is very difficult to secure the neural stem cells (Olanow C. W., Kordower J. H., Freeman T. B. (1996), Fetal nigral transplantation as a therapy for Parkinson's disease. Trends Neurosci. 19, 102-109).

It is a well-established theory in stem cell biology that stem cells require specific cellular microenvironments, or niches, for their culture. As techniques for selectively culturing neural stem cells, neurosphere formation, low-density culture, high-density culture, etc. have been reported. But, it is known that it is difficult to expand the cells in an undifferentiated state in large-scale culture.

Although many researchers have attempted large-scale culture of stem cells, human adult neural stem cells are particularly difficult to culture in vitro and have limited ability to proliferate. For this reason, studies on the human adult neural stem cells are at a standstill.

Soybean is widely used as an ingredient in many foods and has been reported to show anti-cancer, anti-oxidant, anti-inflammatory, anti-lipidemic and estrogen-like effects. Recently, it has been reported that soybean also has learning- and memory-enhancing effects. Soybean contains many phytochemicals, including isoflavones and saponins. Also, isoflavones have been reported to exhibit memory-enhancing, anti-inflammatory and phytoestrogenic effects.

In contrast, soyasaponins have anti-colitic, anti-tumor, hepatoprotective and estrogen-like effects. Soyasapogenol B, which is a metabolite of soyasaponin I, has been reported to inhibit proliferation of human breast cancer cells. However, it is not yet clear whether soyasaponin I has learning- and memory-enhancing effects and what effect it may have on neural stem cells.

Under this background, the inventors of the present invention have made consistent efforts to develop a method for promoting differentiation and proliferation of neural stem cells or neural precursor cells for regeneration of nervous systems and large-scale production of neural stem cells for transplantation. As a result, they have found that soyasaponin I is capable of not only promoting differentiation and proliferation of neural stem cells or neural precursor cells but also recovering damaged nervous systems and have completed the present invention.

### DETAILED DESCRIPTION OF THE INVENTION

### Technical Problem

An object of the present invention is to provide a composition for promoting differentiation or proliferation of neural stem cells or neural precursor cells, which contains soyasaponin I.

Another object of the present invention is to provide a composition for culturing cells, which contains the composition.

Still another object of the present invention is to provide a pharmaceutical composition for preventing or treating neurological diseases by promoting differentiation or proliferation of neural stem cells or neural precursor cells, which contains the composition.

Still another object of the present invention is to provide a health functional food for preventing or improving neurological diseases by promoting differentiation or proliferation of neural stem cells or neural precursor cells, which contains the composition.

### Technical Solution

In order to achieve the above objects, in an aspect, the present invention provides a composition for promoting differentiation or proliferation of neural stem cells or neural precursor cells, which contains soyasaponin I.

In the present invention, the term “soyasaponin I” refers to a compound represented by Chemical Formula 1:

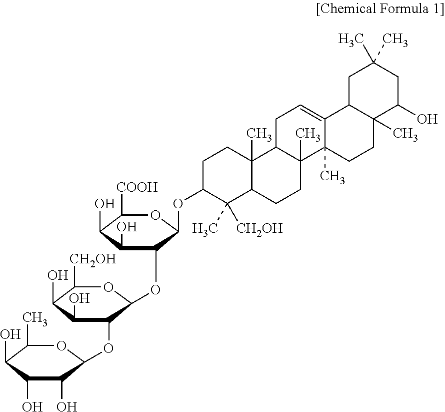

In an exemplary embodiment, the soyasaponin I of the present invention may be prepared to according to a general chemical synthesis method.

In another exemplary embodiment, the soyasaponin I of the present invention may be extracted from soy, although not limited thereto.

In another exemplary embodiment, the soybean may be one of bean, germinated soybean or soybean sprout, although not limited thereto. The soybean to be used may be purchased on the market or cultivated and harvested.

In the present invention, the term “extract” refers to a liquid obtained by immersing a particular substance in various solvents and conducting extraction at room temperature or elevated temperature for a predetermined time, a solid obtained by removing the solvent from the liquid, and so forth.

The solvent is not particularly limited as long as it allows the preparation of an extract having the effect of the present invention of promoting differentiation or proliferation of neural stem cells neural precursor cells. Specifically, water, a polar solvent or a nonpolar solvent may be used and. More specifically, water, a C₁-C₄ lower alcohol (e.g., methanol, ethanol, propanol, butanol, etc.), a mixture solvent thereof, etc., may be used. Most specifically, methanol or a mixture solvent thereof may be used.

A method for obtaining the extract is not particularly limited as long as it allows the preparation of an extract having the effect of promoting differentiation or proliferation of neural stem cells neural precursor cells. For example, crushed soybean may be extracted using a polar solvent such as water or a C₁-C₄ lower alcohol such as methanol, ethanol, etc., about 5-30 times, specifically about 10-20 times of volume based on the dry weight of the soybean, or a mixture solvent thereof with a mixing ratio of about 1:0.1-1:10. The extraction may be performed at a temperature of 20-100° C., specifically 60-100° C., for about from 1 hour to 4 days, by hot water extraction, cold precipitation extraction, reflux condensation extraction, ultrasonic extraction, etc.

However, the extract is not limited thereto as long as can exhibit the effect of the present invention of promoting differentiation or proliferation of neural stem cells neural precursor cells and, in addition to the extract, a diluate or concentrate of the extract, a product obtained by drying the extract, or a crude purified product, purified product or fraction thereof may be included.

In the present invention, the term “fraction” refers to a product obtained by isolating specific components or groups from the extract containing soyasaponin I by fractionation.

The content of the extract containing soyasaponin I or a fraction thereof in the composition of the present invention may be 0.0001-99.9 wt %, more specifically 0.01-50 wt %, based on the total weight of the final composition, although not particularly limited thereto.

In the present invention, the term “neural stem cell” or “neural precursor cell” may be used interchangeably. In some contexts, a cell derived from a neural stem cell may be called a neural precursor cell. Also, the term neural precursor cell includes a “neural progenitor cell”.

In an exemplary embodiment, the neural stem cell or neural precursor cell that can be used in the present invention may be derived from an embryonic stem cell isolated from an early-stage embryo of a mammal, an embryonic germ cell isolated from a primordial germinal cell in the embryonic period or a multipotent adult stem cell isolated from an adult organism. The multipotent adult stem cell may be any multipotent adult stem cell which is derived from the cord blood, adult tissues such as bone marrow, adipose and brain tissues, or blood. In addition, a stem cell prepared by gene transduction, nuclear substitution or fusion of the embryonic stem cell, embryonic germ cell or adult stem cell is included.

In the present invention, the term “differentiation” refers to a process by which a cell in the early stage acquires special properties of each tissue. By the composition according to the present invention, a neural stem cell or neural precursor cell differentiates into a neuron which secretes a neurotransmitter suitable for a particular site.

The inventors of the present invention have identified that the composition containing soyasaponin I according to the present invention promotes differentiation of neural stem cells or neural precursor cells using memory-deficient model rats and embryo-derived neural precursor cells.

The “proliferation” of neural stem cells is a process different from the differentiation. Although many researchers have attempted large-scale culturing of stem cells neural stem cells for transplantation of the stem cells, human adult neural stem cells are particularly difficult to culture in vitro and have limited ability to proliferate. For this reason, studies on the human adult neural stem cells are at a standstill. In addition, most of the proliferated and transplanted neural stem cells die in one year after the transplantation. Accordingly, for development of a cell therapy agent using stem cells, a method for effectively proliferating neural stem cells while maintaining their stem cell capability (stemness) and preventing aging as much as possible.

As a result of consistent studies, the inventors of the present invention have identified that the composition containing soyasaponin I according to the present invention promotes proliferation of neural stem cells or neural precursor cells using memory-deficient model rats and embryo-derived neural precursor cells (Experimental Examples 2 and 3).

Accordingly, since the composition containing soyasaponin I according to the present invention has a remarkable effect of promoting proliferation of neural stem cells or neural precursor cells, it may be usefully used for large-scale culturing of stem cells and regeneration of nervous systems using the same.

In another aspect, the composition according to the present invention may be used as a composition for culturing cells for promoting differentiation or proliferation of neural stem cells or neural precursor cells. The composition for culturing cells may be used to obtain neural stem cells in large quantity in order to treat various diseases associated with the deficiency or damage of neurons. For cell therapy, it may be treated to the neural stem cells or neural precursor cells isolated immediately before transplantation.

The composition for culturing cells may contain soyasaponin I, a plant extract containing soyasaponin I or a fraction alone, or it may further contain another proliferation or differentiation inducing agent. The proliferation or differentiation inducing agent that can be further contained may be any one known as a proliferation or differentiation inducing agent of neural stem cells or neural precursor cells. Examples may include FGF, EGF, retinoic acid, etc., although not limited thereto.

When the composition is injected to a culture of neural stem cells or neural precursor cells, change occurs in secretory proteins such as cell survival- and proliferation-related factors and transcription factors. As a result, the cellular activity is altered and, in particular, cell survival and proliferation are activated. Accordingly, the stem cells that have been produced in large scale due to improved proliferation with the aid of the composition according to the present invention for culturing cells may be transplanted into a disease site as a cell therapy agent in order to promote regeneration of neurons and effectively treat neurological diseases.

In another aspect, the composition containing soyasaponin I according to the present invention may be used as a pharmaceutical composition for preventing or treating neurological diseases. The pharmaceutical composition may directly promote proliferation of isolated or endogenous neural stem cells or neural precursor cells.

The neurological diseases may include dementia, Parkinson's disease, Alzheimer's disease, Pick's disease, Huntington's disease, epilepsy, stroke, ischemic brain disease, degenerative brain disease, memory impairment, traumatic central nervous system disease, spinal cord injury, peripheral nerve injury, amyotrophic lateral sclerosis, peripheral nerve disease, behavioral disorder, developmental disorder, mental retardation, Down's syndrome or schizophrenia, although not limited thereto.

The neurological disease may be caused by insufficient number of neurons, cell damage, apoptosis, etc.

In the present invention, the term “prevention” may mean any action of preventing or delaying the onset of neurological diseases by administering the composition for preventing or treating neurological diseases according to the present invention to a subject.

In the present invention, the term “treatment” may mean any action of ameliorating or improving the symptoms of neurological diseases by administering the composition of the present invention to a subject suspected to have the disease.

In the present invention, the term “improvement” may mean any action of decreasing parameters related to a condition to be treated, e.g., amelioration of the severity of symptoms.

The pharmaceutical composition of the present invention may be used either alone or in combination with another drug known to be effective for neurological diseases. It may be prepared as a single-dosage form using a pharmaceutically acceptable carrier or excipient or may be contained in a multiple-dosage container.

In the present invention, the term “pharmaceutically acceptable carrier” may mean a carrier or a diluent which that does not inhibit the biological activity and characteristics of the administered compound without irritating an organism. The carrier that can be used in the present invention is not particularly limited and any pharmaceutically acceptable carrier commonly used in the art may be used. Non-limiting examples of the carrier may include saline, sterilized water, Ringer's solution, buffered saline, albumin injection solution, dextrose solution, maltodextrin solution, glycerol, ethanol, etc. They may be used either alone or in combination of two or more.

And, if necessary, it may further contain other commonly used additives such as an anti-oxidant, a buffer, a bacteriostat, etc., and may be formulated into an injectable formulation such as aqueous solution, suspension, emulsion, etc. a pill, a capsule, a granule, a tablet, etc., by further adding a diluent, a dispersant, a surfactant, a binder, a lubricant, etc.

The pharmaceutical composition of the present invention may contain a pharmaceutically effective amount of soyasaponin I. In the present invention, the term “pharmaceutically effective amount” refers to an amount sufficient to treat a disease with a reasonable benefit/risk ratio applicable to medical treatment, and the effective amount may be determined considering various factors such as the particular disease to be treated and severity thereof, age and sex of the subject, drug activity, sensitivity to the drug, administration time, administration route, excretion rate, treatment period, drug(s) used in combination and other factors well-known in the medical field. The pharmaceutical composition of the present invention may be administered either alone or in combination with another therapeutic agent sequentially or simultaneously. Also, it may be administered as either single-dosage forms or multiple-dosage forms. It is important to administer an amount that can achieve the maximum effect with the minimum amount without inducing side effects, considering the above-described factors, which can be easily determined by those skilled in the art.

In the present invention, the term “administration” refers to introduction of the pharmaceutical composition of the present invention to a patient by any suitable method. The composition of the present invention may be administered via various administration routes including oral and parenteral routes as long as the composition can reach the target tissue.

The administration method of the pharmaceutical composition according to the present invention is not particularly limited and a method commonly employed in the related art may be used. As non-limiting examples of the administration method, the composition may be administered orally or parenterally. The pharmaceutical composition according to the present invention may be prepared into various formulations depending on the intended administration method.

The administration dose of the pharmaceutical composition of the present invention may be, for example, 1-20 mg/kg, more specifically 1-10 mg/kg, for a mammal including humans. A daily dosage of the composition of the present invention may be administered once daily or may be divided into smaller doses and administered several times a day.

The inventors of the present invention have identified that the composition containing soyasaponin I enhances learning and memory by exhibiting the neuronal regeneration promoting effect of soyasaponin I and can prevent or treat neurological diseases using memory-deficient model rats and embryo-derived neural precursor cells (Experimental Example 1).

In another aspect, the present invention provides a method for preventing or treating neurological diseases, which includes administering the composition for preventing or treating neurological diseases to a subject.

In the present invention, the term “subject” may mean any animal, including human, in which a neurological disease has occurred or is likely to occur. In addition to human, the animal may be a mammal such as cattle, horses, sheep, pigs, goats, camels, antelopes, dogs, cats, etc., requiring treatment of similar symptoms, although not limited thereto.

Specifically, the method for preventing or treating neurological diseases according to the present invention may include administering a pharmaceutically effective amount of the composition to a subject in which a neurological disease has occurred or is likely to occur.

In the present invention, the term “administration” refers to introduction of the pharmaceutical composition of the present invention to a patient by any suitable method. The composition of the present invention may be administered via various administration routes including oral and parenteral routes as long as the composition can reach the target tissue.

In the present invention, the “pharmaceutically effective amount” means an amount sufficient to treat a disease with a reasonable benefit/risk ratio applicable to medical treatment, and a suitable total daily dosage of the composition may be adequately determined by a physician within the scope of proper medical judgment. In general, a daily dosage of 0.001-1000 mg/kg, specifically 0.05-200 mg/kg, more specifically 0.1-100 mg/kg, may be administered at once or may be divided into smaller doses and administered several times a day. However, for the purpose of the present invention, a specific pharmaceutically effective amount for a particular patient will vary depending on various factors such as the particular composition, including the kind and extent of response desired to be achieved and the presence of another drug used together with the composition, the age, body weight, general physical conditions and sex of the patient, diet, administration time, administration route, the excretion rate of the composition, treatment period and other similar factors well-known in the medical field.

In another aspect, the present invention provides a food composition for preventing or improving neurological diseases.

Since soy containing the soyasaponin I has long been used as a food source with proven safety, the composition containing soyasaponin I can be prepared into food and consumed to prevent or improve neurological diseases.

The food composition of the present invention may further contain a sitologically acceptable carrier.

The food composition of the present invention may be in the form of pill, powder, granule, infusion, tablet, capsule, liquid, etc. The composition containing soyasaponin I of the present invention may be added to various foods, e.g., various drinks, gums, teas, vitamin complexes, supplementary health foods, etc., without particular limitation. The food composition may further contain other ingredients as long as the effect of preventing or improving neurological diseases is not negatively affected. The additional ingredients are not particularly limited. For example, various herbal extracts, sitologically acceptable auxiliary food additives, natural carbohydrates, etc. may be further included as in common foods.

In the present invention, the term “auxiliary food additive” refers to an ingredient that can be added supplementarily to foods. It is added to prepare each formulation of a health functional food and may be adequately selected by those skilled in the art. Examples of the auxiliary food additive include various nutrients, vitamins, minerals (electrolytes), synthetic or natural flavors, colorants, fillers, pectic acid and salts thereof, alginic acid and salts thereof, organic acids, protective colloid thickeners, pH control agents, stabilizers, antiseptics, glycerin, alcohols, carbonating agents used in carbonated drinks, etc. However, the auxiliary food additive of the present invention is not limited to the above-described examples.

Examples of the natural carbohydrate include monosaccharides such as glucose, fructose, etc., disaccharides such as maltose, sucrose, etc., polysaccharides such as dextrin, cyclodextrin, etc. and sugar alcohols such as xylitol, sorbitol, erythritol, etc. In addition, a natural sweetener (thaumatin, stevia extract (e.g., rebaudioside, glycyrrhizin, etc.)) or a synthetic sweetener (saccharin, aspartame, etc.) may be advantageously used.

The food composition of the present invention may be contained in a health functional food. In the present invention, the term “health functional food” refers to a food prepared or processed into tablet, capsule, powder, granule, liquid, pill, etc. using raw materials or ingredients with useful functions for the human body. As used herein, the term functional means a useful effect for human health, such as structural or functional regulation of nutrients, physiological action, or the like. The health functional food of the present invention may be prepared according to a method commonly employed in the art, and commonly used raw materials and ingredients may be added when preparing the health functional food. Since it uses food ingredients as raw materials, the health functional food lacks side effects which may occur when a drug is taken for a long period of time and may have excellent portability.

When the composition of the present invention is included in a health functional food, the composition may be added alone or together with another health functional food or health functional food ingredient(s), according to a commonly employed method. The amount of the active ingredient may be determined appropriately depending on the purpose of use (prevention, health improvement, or therapeutic intervention). In general, the composition containing soyasaponin I of the present invention is included in an amount of 1-10 wt %, specifically 5-10 wt %, based on the weight of the health functional food. However, the content may be lower than the above-described range when the health functional food is ingested for a long period of time for promoting health and hygiene.

The kind of food is not specially limited. Examples of the food to which the composition can be added include meat, sausage, bread, chocolate, candy, snack, confectionery, pizza, ramen, other noodles, gum, dairy products including ice cream, soup, beverage, tea, drink, alcoholic beverage, vitamin complex, etc. All health functional foods in the context of common sense are included.

Since the composition containing soyasaponin I according to the present invention has an effect of promoting differentiation or proliferation of neural stem cells or neural precursor cells, it may be usefully used as a composition for cells in order to culture neural stem cells for laboratory use or transplantation in large scale. Also, it may be used to promote differentiation into neurons suitable for the transplantation site and regeneration of damaged neurons.

Accordingly, the composition containing soyasaponin I according to the present invention may be used to prevent, improve and treat various neurological diseases.

### BRIEF DESCRIPTION OF THE DRAWINGS

FIGS. 1A-1D show the effect of soyasaponin I in memory-deficient model rats. FIG. 1A describes an experimental procedure, FIG. 1B and FIG. 1C show results of a Y-maze test (spontaneous alteration and number of total entry), and FIG. 1D shows a result of a passive avoidance test.

FIGS. 2A-2B show the effect of administration of soyasaponin I on promoted proliferation of neural precursor cells in memory-deficient model rats. FIG. 2A shows a result of staining the cells expressing the neuron proliferation marker BrdU with PI (Sham: negative control, Control: memory-deficient model (positive control), Soya-I: memory-deficient model rats administered with soyasaponin I), and FIG. 2B shows the number of the cells expressing BrdU per hippocampal slice.

FIGS. 3A-3E show the effect of administration of soyasaponin I on change in neuronal cell types and microglia activation in memory-deficient model rats. FIG. 3A shows an immunostaining result of neurons with specific markers GAD67 (GABAergic neurons), VGluT1 (glutamatergic neurons), ChAT (cholinergic neurons) and OX42 (reactive microglia), FIG. 3B shows the number of GAD67-positive cells per hippocampal slice, FIG. 3C shows the number of VGluT1-positive cells per hippocampal slice, FIG. 3D shows the number of ChAT-positive cell per hippocampal slice, and FIG. 3E shows the OX42 intensity compared with the negative control (Sham).

FIGS. 4A-4G show the results of animal testing conducted 2 weeks after administration of soyasaponin I to memory-deficient model rats. FIG. 4A describes an experimental procedure, FIG. 4B and FIG. 4C show results of a Y-maze test (spontaneous alteration and number of total entry), FIG. 4D shows a result of a passive avoidance test, and FIG. 4E, FIG. 4F and FIG. 4G show a result of a Morris water maze test.

FIGS. 5A-5E show the effect of administration of soyasaponin I on promoted differentiation of neural precursor cells which have newly proliferated 4 weeks after the administration of soyasaponin I to memory-deficient model rats. FIG. 5A shows the expression of a mature neuron marker (NeuN) and cell subtype markers (VGluT1 and GAD67), FIG. 5B shows the number of BrdU-positive cells per hippocampal slice, FIG. 5C shows the number merged cells per hippocampal slice, FIG. 5D shows the number of ChAT-positive cells per hippocampal slice, and FIG. 5E shows the number of ChAT-positive cells per hippocampal slice.

FIGS. 6A-6B show the effect of soyasaponin I on promoted proliferation of neural precursor cells, and FIGS. 6C-6H show the effect of soyasaponin I on promoted differentiation of neural precursor cells and neurite outgrowth.

FIGS. 7A-7C show the effect of soyasaponin I on differentiation of neurons.

### EXAMPLES

Hereinafter, the present invention will be described in detail through examples. However, the following examples are for illustrative purposes only and the scope of the present invention is not limited by the examples.

### Example 1

### Preparation of Materials

Dulbecco's modified eagle medium-F12 media, Ca⁺²/Mg⁺²-free Hank's balanced salt solution (HBSS), basic fibroblast growth factor (bFGF), trypsin-ethylenediaminetetraacetic acid, secondary antibodies and L-glutamine were purchased from Invitrogen (Carlsbad, Calif., USA). Primary antibodies were purchased from Abcam (Cambridge, UK), Chemicon (Billerica, Mass., USA) and Serotec (Kidlington, UK). Secondary antibodies were purchased from Jackson (West Grove, Pa., USA). Ibotenic acid (IBO), poly-L-ornithine, progesterone, D-(+)-glucose, putrescine, apotransferrin, insulin, fibronectin, 4-(2-hydroxyethyl)-1-piperazineethanesulfonic acid, phenol red, a radioimmunoprecipitation assay buffer, phosphate-buffered saline (PBS), a phosphatase inhibitor cocktail, a protease inhibitor cocktail, dimethyl sulfoxide, paraformaldehyde (PFA) and Tween 80 were purchased from Sigma (St. Louis, Mo., USA).

Soyasaponin I (purity ≧95%) was extracted from soy as reported by Chang et al. (2009). All other materials were obtained from normal commercial sources.

### Example 2

### Generation of Memory-Deficient Rat Model and Administration of Soyasaponin I

(1) Preparation of Animals

Male laboratory rats (200-250 g) were obtained from Orient Animal Breeding Center (a branch of Charles River Laboratories, Gyeonggi-do, Korea). The rats were housed 4 or 5 per cage for at least 1 week of accommodation, allowed access to water and food ad libitum, and maintained under a constant temperature (20-23±1° C.) and humidity (60±10%) condition and a 12 h/12 h light/dark cycle (light on 07:00-19:00). After the surgical procedure, the rats were housed 2 or 3 per cage to avoid social stress. All experiments were performed in accordance with the NIH and the Kyung Hee University guidelines for Laboratory Animals Care and Use and approved by the Committee for the Care and Use of Laboratory Animals in the College of Pharmacy, Kyung Hee University.

(2) Generation of Memory-Deficient Rat Model

Ibotenic acid is a neurotoxin present in mushrooms and is known to cause powerful damage to neurons when directly injected into the brain of mice or rats. In particular, because senile plaques and neuronal cell death appear in the entorhinal cortex and hippocampus in early Alzheimer's disease patients (Ueki A et al. J Neurol Sci, 1994), memory-deficient model rats were generated by injecting ibotenic acid (IBO) into the entorhinal cortex. The surgical protocol followed Heo H et al.'s method (J Ethnopharmacol, 2009) with some modifications. In the model animals, the injection of ibotenic acid induces cell death not only in the entorhinal cortex but also in the CA and DG regions of the hippocampus and impairs hippocampus-related learning and memory abilities.

The 6-week-old rats were anesthetized with equithesin (350 mM pentobarbital sodium, 250 mM chloral hydrate, 85 mM MgSO₄ and 40% propylene glycol in 10% ethanol, 2 mL/kg). To induce cell death in the dentate gyrus and hippocampus in addition to the entorhinal cortex, ibotenic acid (1.5 μL per animal, 1 mg/mL) was injected into the entorhinal cortex as follows. The rat was placed in a stereotaxic device (Stoelting Co., Wood Dale, Ill., USA) with the incisor bar 3.4 mm below the interaural line. The needle was positioned 10° right to the midsaggital plane. Then, ibotenic acid was injected at 3 locations into the entorhinal cortex and medial entorhinal cortex at a rate of 0.35 μL/min (first, AP: 8.4 mm, ML: 4.8 mm, DV: 4.6-4.8 mm; second, AP: 8.4 mm, ML: 4.8 mm, DV: 2.6-2.8 mm; third, AP: 8.8 mm, ML: 3.65 mm, DV: 4.8 mm).

As a normal group (Sham), physiological saline was injected at the same locations instead of ibotenic acid.

(3) Administration of Soyasaponin I

A vehicle (2% Tween 80) was administered to the IBO group and 5, 10 or 20 mg/kg of soyasaponin I was administered to the Soya-I group. The vehicle or soyasaponin I was administered orally (1 mL, once a day) by intubation using an oral feeding needle (oral zonde needle, 9 cm) and no vomiting reflex was observed.

### Experimental Example 1

### Evaluation of Effect of Soyasaponin I on Improvement of Learning and Memory Abilities

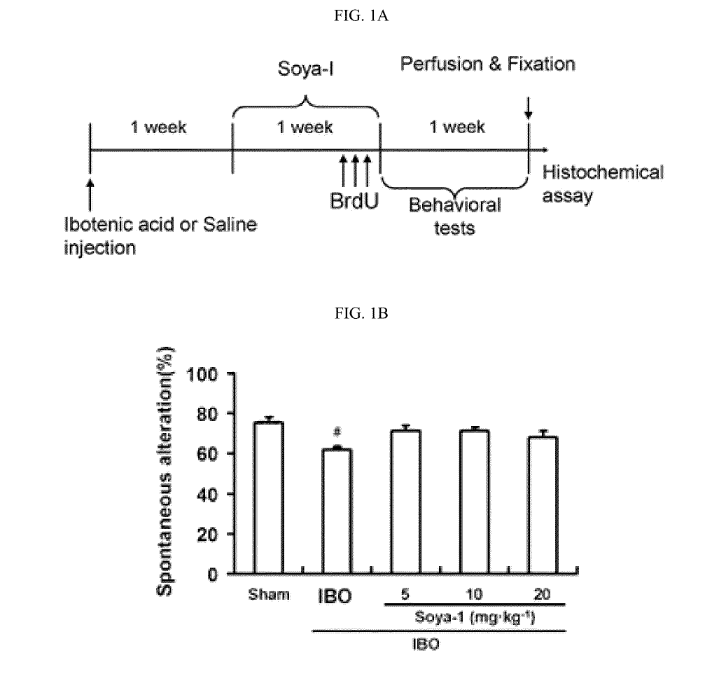

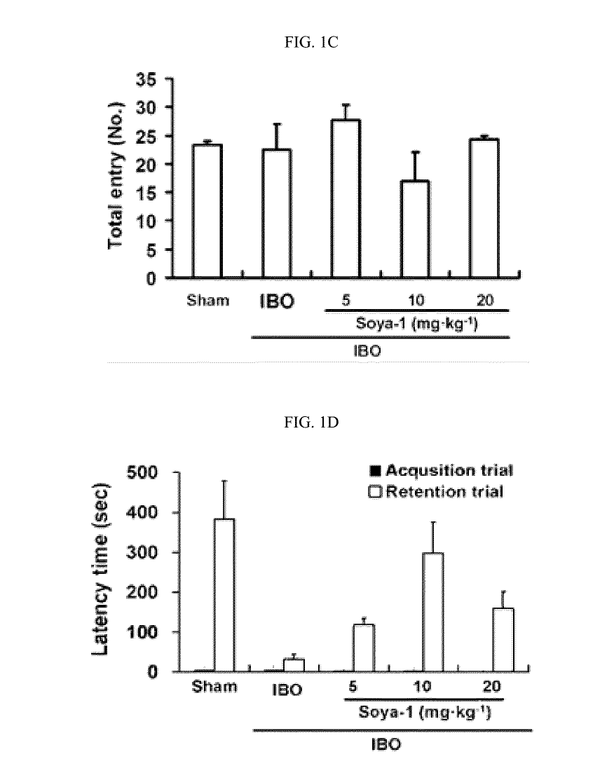

In order to investigate whether soyasaponin I improves the impaired learning and memory abilities of the memory-deficient model rats generated in Example 2 by promoting neuronal regeneration in the hippocampus, a Y-maze test, a passive avoidance, a Morris water maze test and an immunohistochemical assay were conducted as described in FIG. 1A.

(1) Y-Maze Test

The Y-maze test was performed first among the behavioral tests referring to Heo H et al. (J Ethnopharmacol, 2009).

The maze was made of black-colored acryl and was positioned at equal angles. The rats were separated by groups (group 3: n=12, group 2: n=12, group 1: n=5-6) and were accustomed in the Y-maze room for 30 minutes. The rats were placed at the end of an arm of the Y-maze and allowed to move freely through the maze and to enter as many arms they like for 8 minutes. Arm entry was recorded when the hind paws were completely placed in the arm. Consecutive entry into 3 arms in an alternative order was defined as successive entries on overlapping triplet sets, and alternation percentage was calculated as the ratio of actual to possible alternations (defined as the total number of arm entries minus 2), multiplied by 100.

The result is shown in FIG. 1B and FIG. 1C.

(2) Passive Avoidance

The passive avoidance was carried out as described in Heo H et al. (J Ethnopharmacol, 2009), with the same number of animals per group as the Y-maze test. A semi-automated system with a shuttle chamber was used. The rats were trained to avoid light by entering a dark chamber through an acryl door when light was turned on. This train was repeated three times a day until the rat had entered the dark chamber within 20 seconds (training trial) for 3-4 days. In this study, all the rats were successfully trained to enter within 20 seconds on the final training day. After performing the acquisition trial on the last day of training, the rats were placed in a lighted chamber and, when they entered the dark chamber, the door was closed manually and an electric foot shock (1 mA) was delivered for 3 seconds through a grid floor. Exactly 24 hours after the acquisition trial, the rats were placed in the lighted chamber again and the latency time to enter the dark chamber was measured for 720 seconds (retention trial).

The result is shown in FIG. 1D.

(3) Morris Water Maze Test

The Morris water maze test was conducted with 13-week-old rats (normal group: n=13, ibotenic acid group: n=13, Soya-I group: n=6) in a white circular stainless pool (160 cm in diameter, 60 cm in height). The pool was filled with water (maintained at 23.0±1.0° C.) to a depth of 50 cm. An invisible platform (15 cm, circular, white) was submerged 1.0 cm below the water surface and placed at the center of the northeast quadrant. Each rat was given one trial a day for 4 days to find the hidden platform. The trial was initiated by placing the rat in the water facing the pool wall, in one of the four quadrants randomly. All the four quadrants were used once every day.

For each trial, the rat was allowed to swim for a maximum of 60 seconds to find the platform and to rest for 1 minute on the platform upon success. The average time for the 4 quadrant tests was defined as the escape latency time per group on the training days. On the last day of the training trial, the rats were subjected to a probe trial in which the platform was removed. The rats were allowed to swim for 60 seconds to search for the location where the platform had been. The swimming time and length of the trial were recorded and monitored using a video camera. Tracking was accomplished by following the trajectory of the white rat, which was indicated by a black point against a white background. The captured video images were analyzed by a video tracking system (EthoVision Water Maze program, Noldus Information Technology, Wageningen, the Netherlands). The analyzed information included the swimming time in the target quadrant and the number of virtual platform crossings to find the removed platform.

(4) Immunohistochemical Analysis—BrdU

After conducting the three behavioral tests, the rats were sacrificed for immunohistochemical analysis.

The brain slices of the test animals of the normal group (Sham), the IBO group and the Soya-I group were immunostained by the method of Heo H et al. (J Ethnopharmacol, 2009) with modifications. The rats were transcardially perfused with 4% PFA in PBS. Following fixation by immersing in 4% PFA in PBS for 3 hours, the brain tissues were cryoprotected in 30% sucrose in PBS, frozen with an optimal cutting temperature (OCT) compound and stored at −80° C.

The brain tissue blocks were cryosectioned through the coronal plane at a thickness of 35 μm. The brain tissue sections were stored at 4° C. in a storing solution (30% glycerol and 30% ethylene glycol in PBS). The stored brain slices were permeabilized in 0.5% Triton X-I00 for 20 minutes and incubated at 37° C. for 30 minutes in 2 N HCl. Then, they were blocked in 15% normal serum and 3% bovine serum albumin (BSA, bio-WORLD, Dublin, Ohio, USA) and 0.1% Triton X-100 for 2 hours under a free-floating condition.

The sections were incubated for 16 hours at 4° C. with primary antibodies against bromodeoxyuridine (BrdU) (Sigma).

Secondary antibodies conjugated with Alexa Fluor 488 (1:1,000, Invitrogen), Cy2 (1:500, Jackson) and Cy3 (1:500, Jackson) were used. The cell nuclei were counterstained with 1 μg/mL propidium iodide (PI, Sigma) for 5 minutes. The immunostained sections were scanned with a confocal laser microscope (LSM510, Carl Zeiss, Oberkochen, Germany).

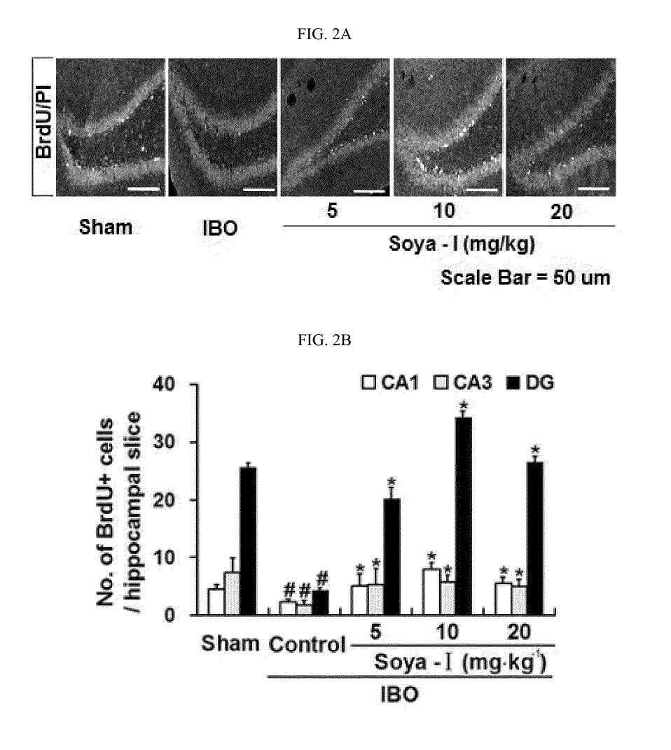

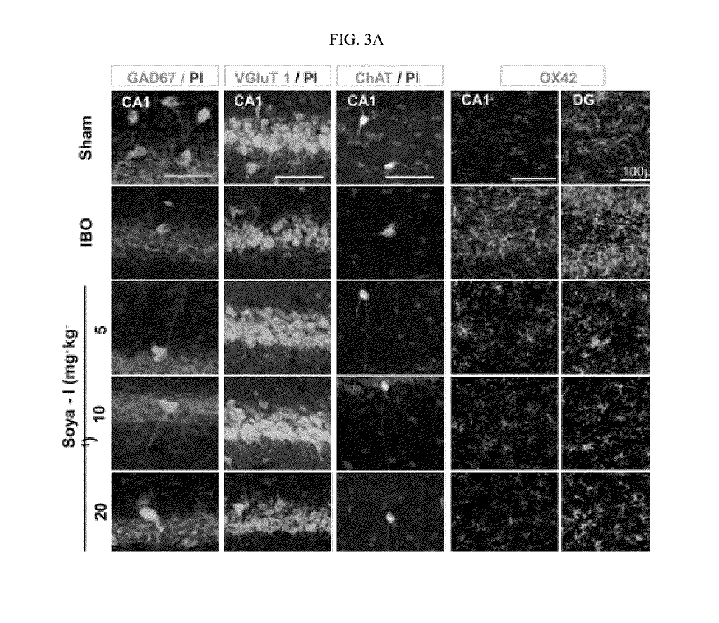

Over 5 animals from each group divided in (1) were used for immunohistochemical analysis. The result is shown in FIG. 2A and FIG. 2B.

(5) Immunohistochemical Analysis—VGluT1, ChAT, GAD67, OX42

Immunostaining was carried out using antibodies against VGluT1 (diluted 1:500, Chemicon), ChAT (1:500, Chemicon), GFAP (1:1,000, Chemicon), tyrosine hydroxylase (TH, 1:500, Chemicon) and OX42 (CD11b/c, 1:250, Serotec) as primary antibodies instead of the anti-BrdU antibodies used in (4). The other process was the same as in (4), except that incubation was performed at 37° C. for 30 minutes in 2 N HCl.

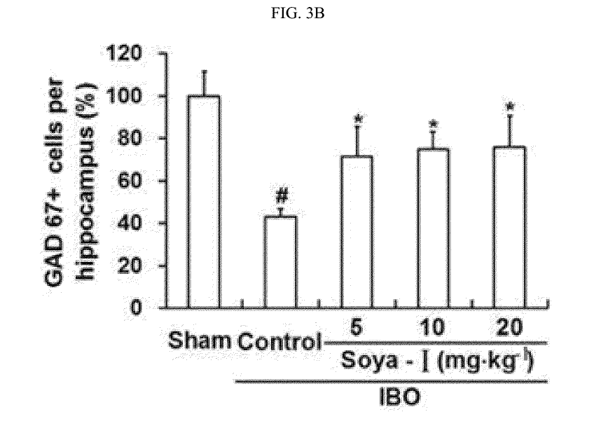

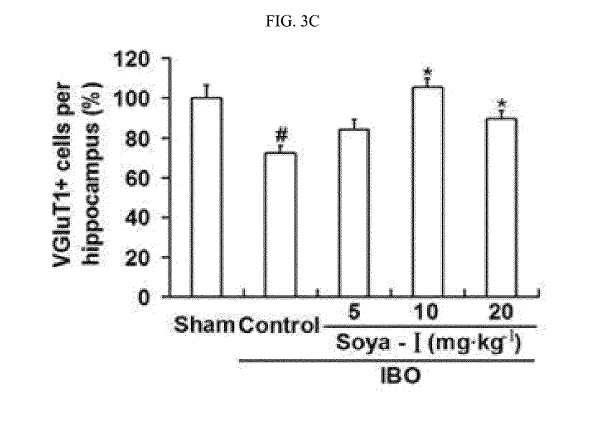

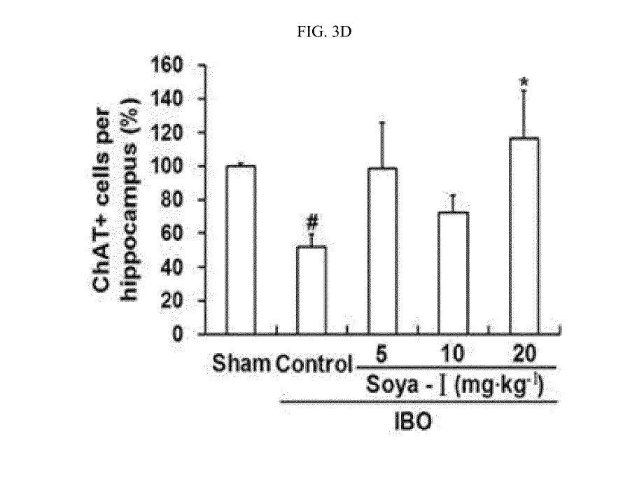

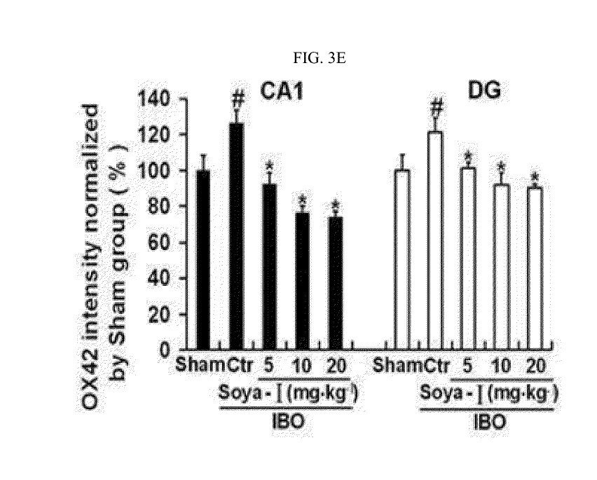

The result is shown in FIGS. 3Aa-3E.

### Experimental Example 2

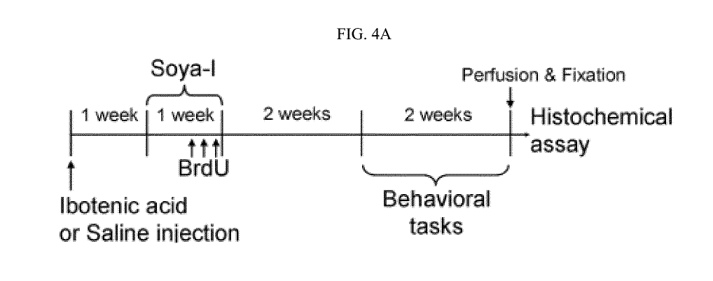

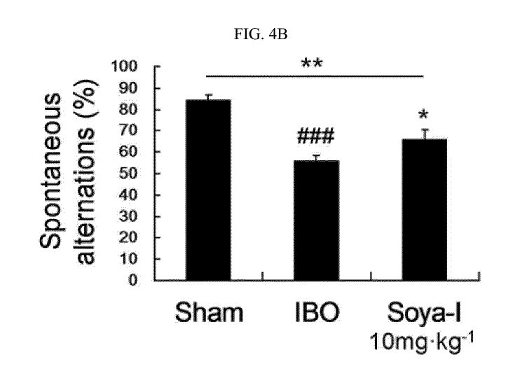

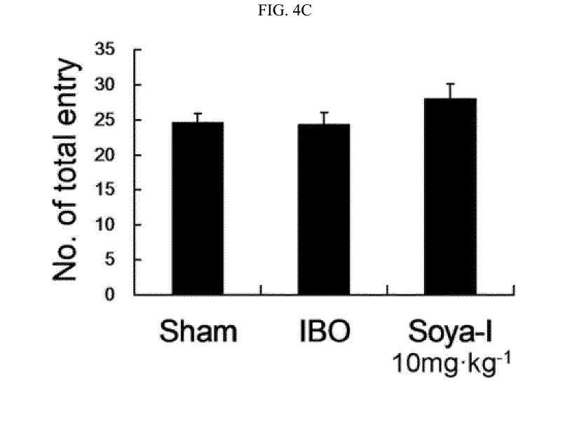

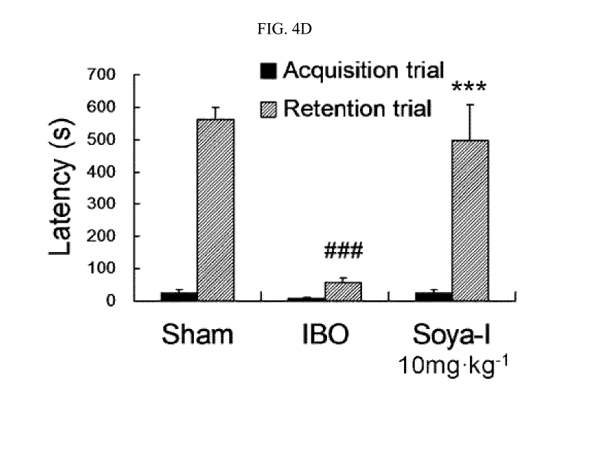

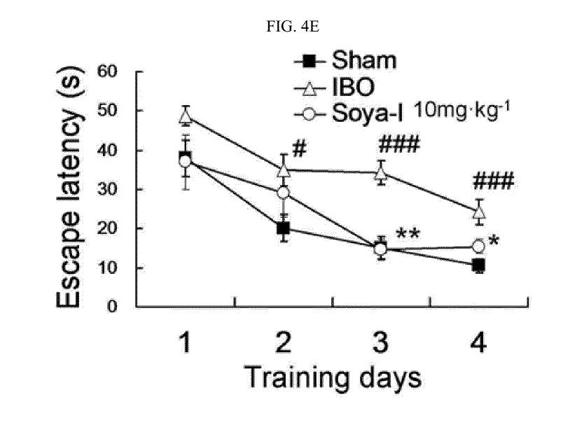

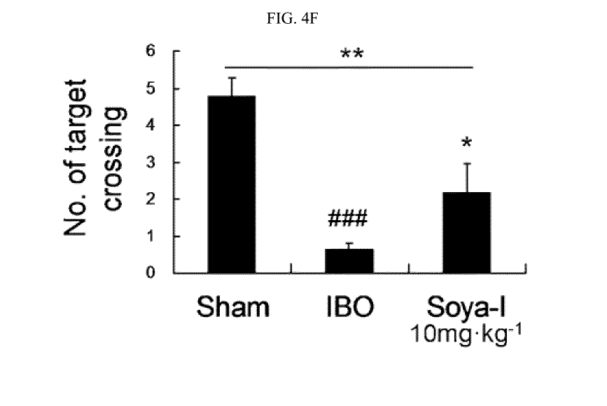

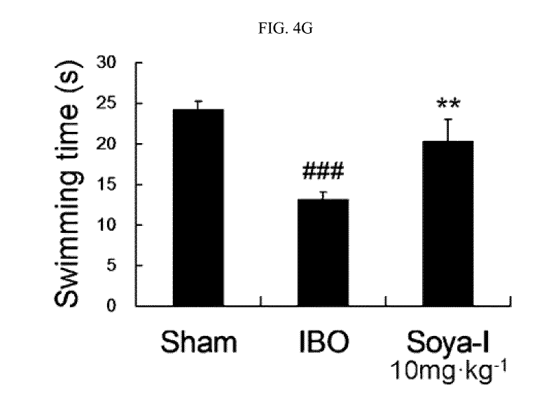

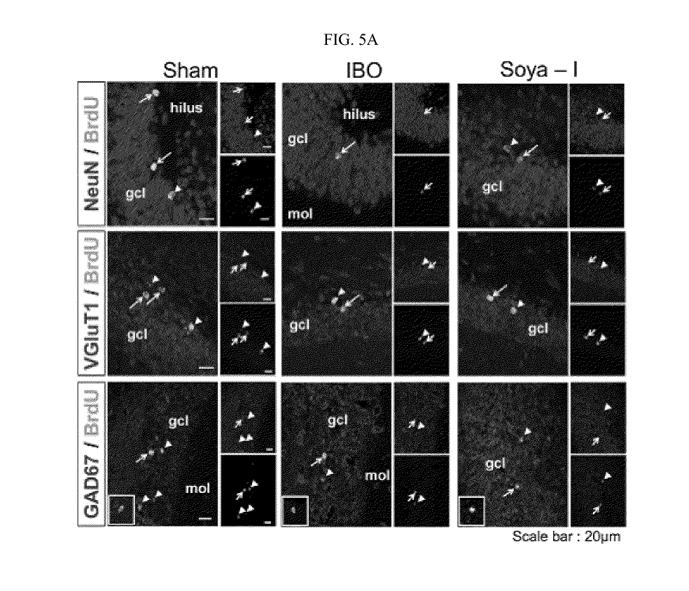

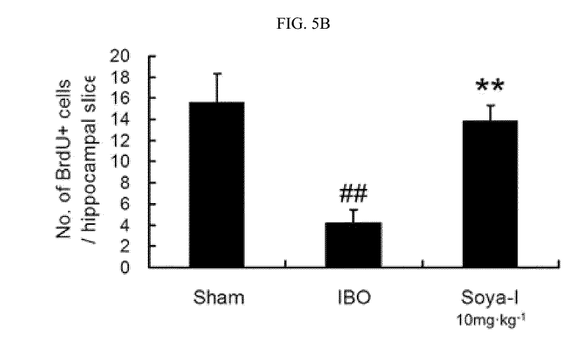

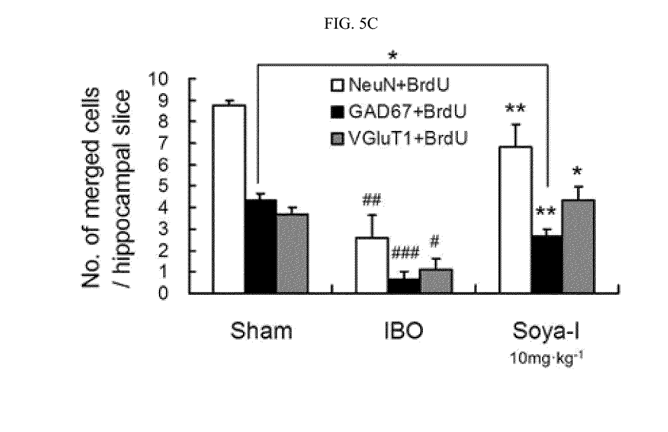

### Behavioral Tests and Immunohistochemical Analysis 2 Weeks after Administration of Soyasaponin I

Whereas the behavioral tests were conducted immediately after the administration of soyasaponin I in Experimental Example 1, behavioral tests including a Y-maze test, a passive avoidance test and a Morris water maze test were carried out 2 weeks after the administration of soyasaponin I for 2 weeks in Experimental Example 2 in order to investigate the effect of promoting proliferation of new-born neural precursor cells. The experimental procedure is described in FIG. 4A.

If newly born neural precursor cells survive, proliferate and get incorporated into the neural network and neuronal cell types are increased as a result thereof, the ability of memory formation can be maintained. Therefore, the behavioral tests were conducted with the time interval in consideration of the period during which the neural precursor cells are newly generated after the administration of soyasaponin I.

(1) Behavioral Tests

The Y-maze test, the passive avoidance test and the Morris water maze test were conducted in the same manner as in Experimental Example 2. The result is shown in FIGS. 4B-4G.

(2) Immunohistochemical Analysis

In order to confirm the survival and differentiation of newly born neural precursor cells 4 weeks after the oral administration of soyasaponin I, it was observed whether the BrdU-expressing cells express a mature neuron marker and cell subtype markers (NeuN, VGluT1, GAD67) simultaneously.

After conducting the 3 behavioral tests, the rats were sacrificed for immunohistochemical analysis. Over 5 animals from each group were used for the immunohistochemical analysis.

Following the same procedure as described in (4) of Experimental Example 2, incubation was performed at 37° C. for 30 minutes in 2 N HCl after permeabilization in 0.5% Triton X-100 for 20 minutes for double staining of BrdU/subtype cell markers (GAD64, VGluT1, NeuN).

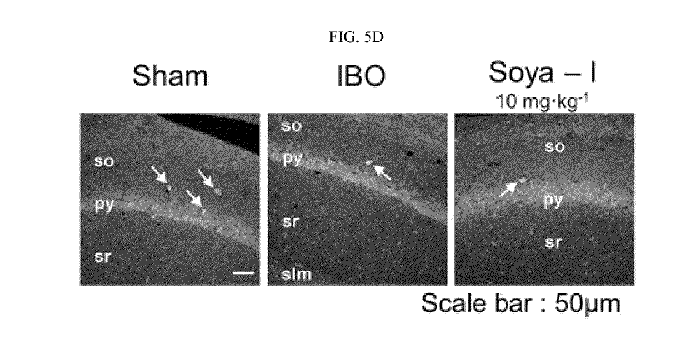

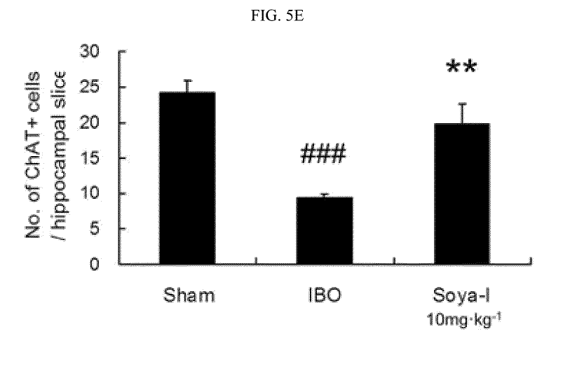

The result is shown in FIGS. 5A-5E.

### Experimental Example 3

### Effect of Soyasaponin I on Differentiation and Proliferation of Embryonic Neural Precursor Cells

In order to investigate whether soyasaponin I has an effect of directly inducing proliferation and differentiation of neural precursor cells, neural precursor cells isolated from the embryo were cultured and subjected to immunoblotting assay and immunocytochemical assay.

(1) Isolation and Culturing of Embryonic Neural Precursor Cells

Pregnant Sprague-Dawley rats purchased from Orient Animal Breeding Center (a branch of Charles River Laboratories, Gyeonggi-do, Korea) were sacrificed by exposing to CO₂ inhalation. Hippocampal neural precursor cells (NPCs) were isolated from the brain of the E16 rat embryo according to the method of Shin M H et al. (Exp Mol Med, 2002) with slight modifications.

Briefly, hippocampi were aseptically dissected from the embryonic forebrain, mechanically dissociated in Ca²⁺/Mg²⁺-free Hank's balanced salt solution (HBSS) and then plated at 7×10⁵ cells/cm² on 60-mm dishes pre-coated with 15 μg/mL poly-L-ornithine and 1 μg/mL fibronectin. The neural precursor cells were cultured in serum-free N2 media supplemented with 10 ng/mL bFGF in 5% CO₂ for 3 days and then detached using 0.05% trypsin-ethylenediaminetetraacetic acid (EDTA). The neural precursor cells were plated on 12-mm cover glasses (Bellco Co., Vineland, N.J., USA) and cultured one more day in N2 media supplemented with 10 ng/mL bFGF in 5% CO₂ (primary culturing).

(2) Immunohistochemical Analysis—Ki67

Following the primary culturing in (1), immunohistochemical analysis was conducted using Ki67 antibodies in order to confirm the effect of soyasaponin I on proliferation of the embryonic neural precursor cells.

First, the neural precursor cells were cultured on 12-mm cover glasses (Bellco Co., Vineland, N.J., USA) for 11 days in bFGF-free N2 media with 2×10⁴ cells/well.

Immunostaining of the cultured neural precursor cells (NPCs) was carried out as previously described (Donaldson J G (2001) Immunofluorescence staining. Curr Protoc Cell Biol Chapter 4: Unit 4.3) with minor modifications.

On the next day, after adding a vehicle (dimethyl sulfoxide) or Soya-I at different concentrations of 0.5, 1 or 2 μM to the media, the cells were fixed with 4% PFA in PBS for 4-15 minutes. The cells were permeabilized with 0.5% Triton X-100 for 5 minutes and blocked with 5% normal serum (a mixture of normal donkey, goat and horse sera) for 1 hour at room temperature.

The cells were incubated with anti-Ki67 (1:500, Abcam) primary antibodies for 1 hour at room temperature. Secondary antibodies conjugated with Cy3, Cy5 (1:700, Jackson) or Alexa 488 (1:1,000, Invitrogen) were used. The cell nuclei were counterstained with 1 μg/mL PI for 5 minutes. The immunostained cells were scanned under a confocal laser microscope (LSM10, Carl Zeiss).

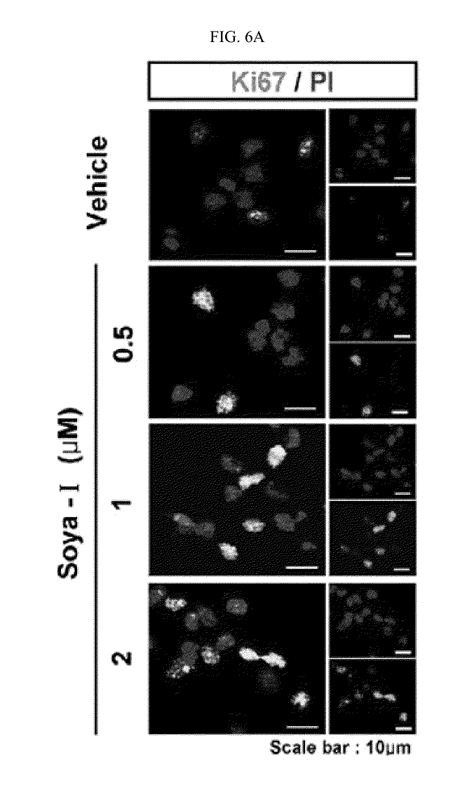

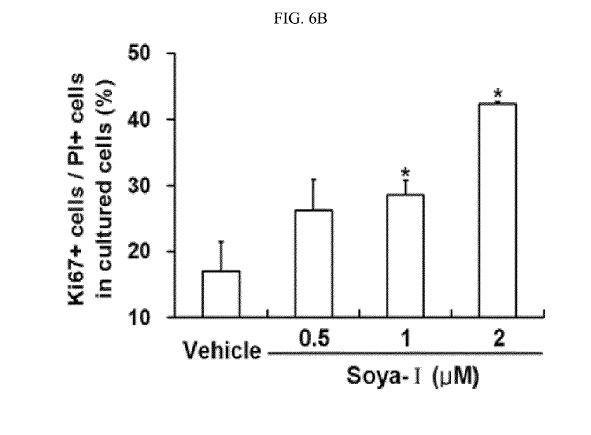

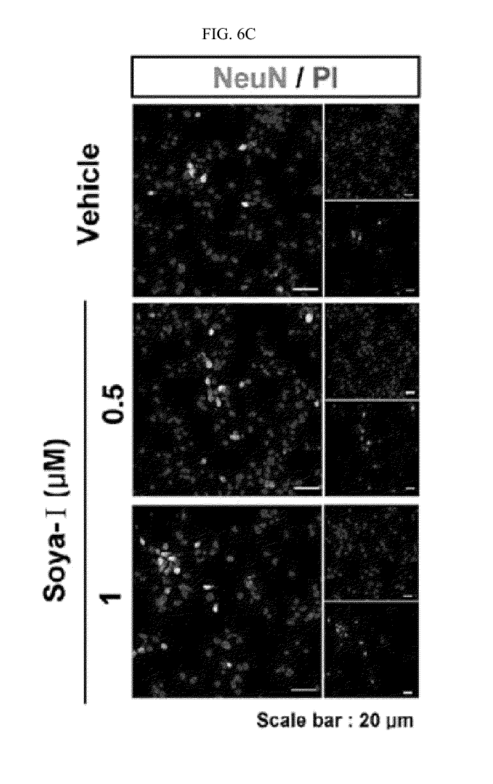

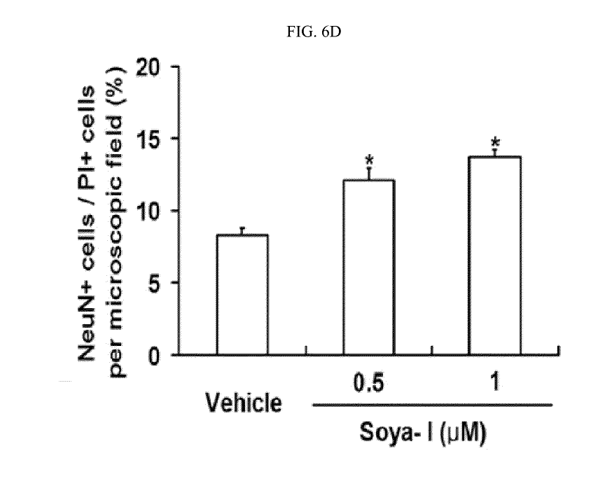

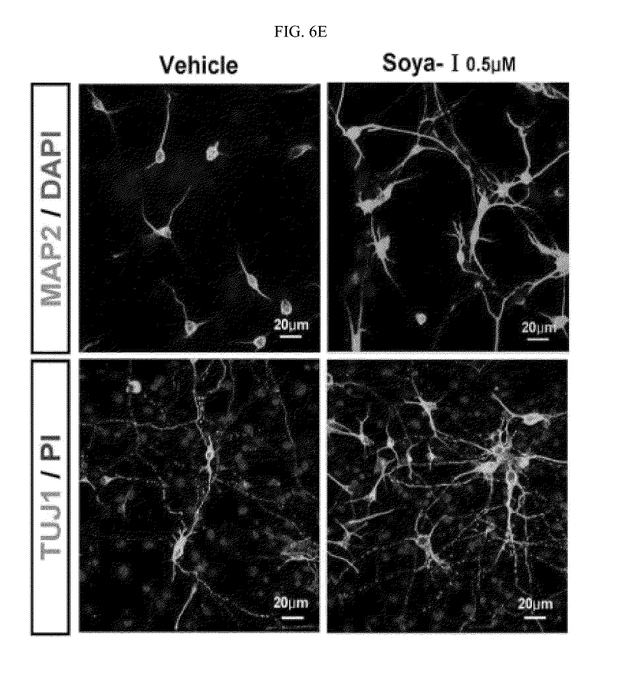

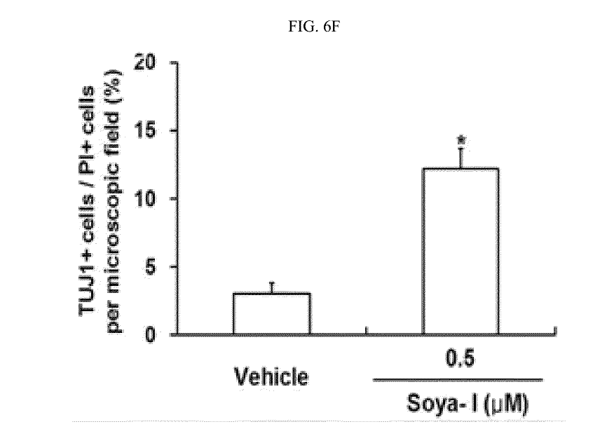

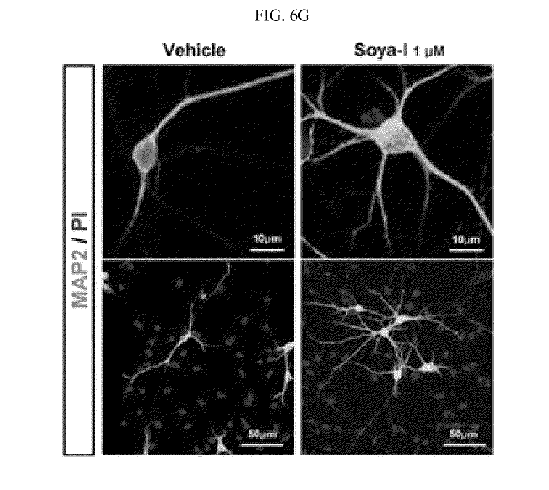

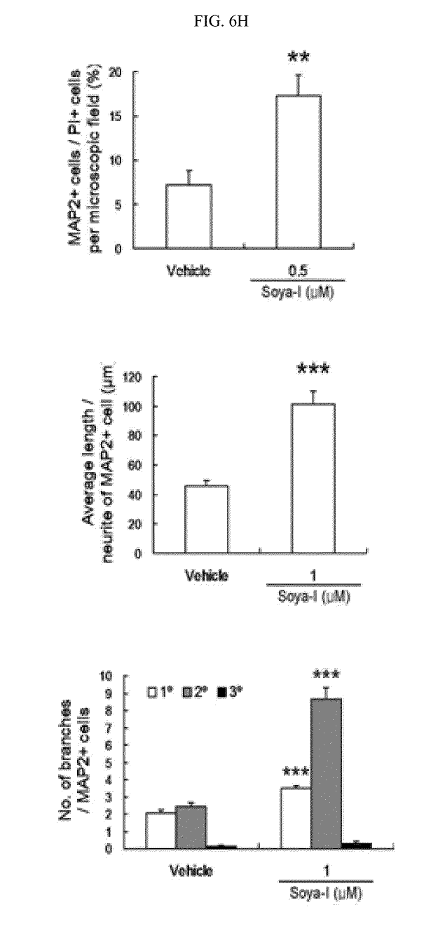

The result is shown in FIG. 6A and FIG. 6B.

(3) Immunohistochemical Analysis—NeuN, GFAP, TUJ1, MAP2

Following the primary culturing in (1), in order to confirm the differentiation of the neural precursor cells and the proliferation of the differentiated neurons, the neural precursor cells were cultured on 12-mm cover glasses (Bellco Co., Vineland, N.J., USA) for 4 days in bFGF-free N2 media with 2×10⁴ cells/well. For double immunostaining of GFAP and NeuN, and TUJ1 and MAP2, the cells were cultured for only 2 days.

The immunostaining was carried out as previously described (Donaldson J G (2001) Immunofluorescence staining Curr Protoc Cell Biol Chapter 4: Unit 4.3) with minor modifications.

On the next day, after adding a vehicle (dimethyl sulfoxide) or soyasaponin I at different concentrations of 0.5, 1 or 2 μM to the media, the cells were fixed with 4% PFA in PBS at 4° C. for 4-15 minutes. The cells were permeabilized with 0.5% Triton X-100 for 5 minutes and blocked with 5% normal serum (a mixture of normal donkey, goat and horse sera) for 1 hour at room temperature.

The cells were incubated with anti-GFAP (1:1,000, Chemicon), anti-NeuN (1:500, Chemicon), anti-MAP2 (1:2,500, Sigma) and anti-TUJ1 (1:2,000, Sigma) primary antibodies for 1 hour at room temperature. The secondary antibodies and scanning method used were the same as in (2).

The result is shown in FIGS. 6C-6H.

(4) Immunoblotting Assay—VGluT1, ChAT, GAD65/67

Following the primary culturing in (1), the cultured hippocampal neural precursor cells were lysed in 80 μL of a cold RIPA buffer containing a protease inhibitor cocktail for immunoblotting assay of VGluT1, ChAT and GAD65/67. After centrifugation for 10 minutes at 13,000×g, the supernatants were divided into Eppendorf tubes and stored at −80° C.

Protein quantification assay was performed using a Bradford protein assay kit (Bio-Rad, Hercules, Calif., USA). For immunoblotting assay, a mixture of a sample loading buffer (Biosesang, Inc., Seoul, Korea) and 20 μg of proteins was boiled at 100° C. for 10 minutes. Denatured proteins were separated by 10% polyacrylamide gel electrophoresis or 2-3 hours at 100 V and transferred to 0.2-μm nitrocellulose membranes for 2-3 hours at 100 V.

The membranes were then washed 3 times with 0.1% Tween-20 in PBS for 15 minutes between each of the following steps: blocking for 1 hour in 5% skim milk, overnight incubation at 4° C. with primary antibodies against VGluT1 (1:400, Chemicon), GAD65/67 (1:500, Chemicon), ChAT (1:400, Chemicon) and β-actin (1:1,000, Santa Cruz, Del., Calif., USA), and incubation for 1 hour at room temperature with secondary antibodies.

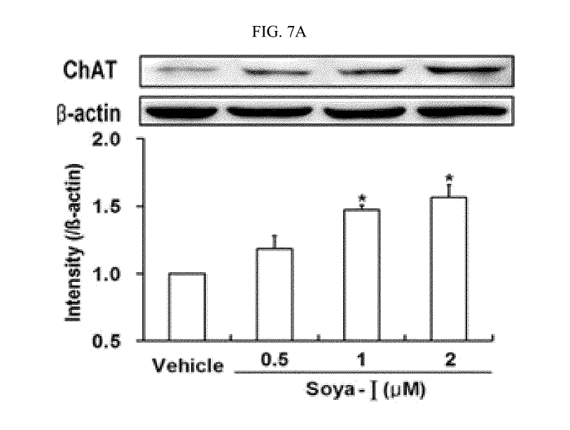

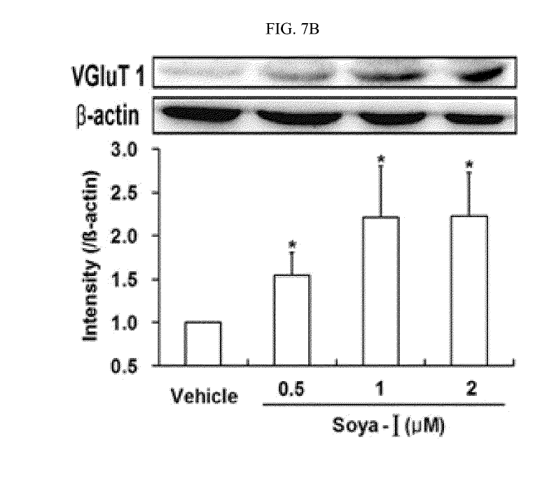

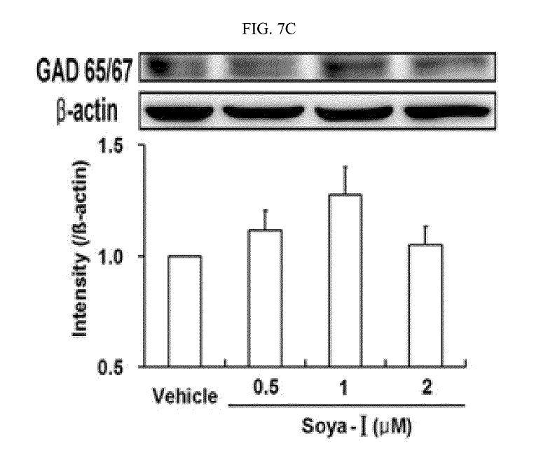

The result is shown in FIGS. 7A-7C.

(5) Quantification and Statistics

Cell counting was performed as reported by Park H J et al. (Phytother Res, 2008). The average length per neurite of the MAP2-positive cells with longer neurites more than double the cell body width was counted from 10-12 confocal laser microscopic fields that were randomly chosen per the cultured cover glass. Primary branches, which directly outgrow from the cell body, were counted when the length of the primary branches was 2 times or longer than the cell body width. All secondary and tertiary branches that come from the primary and secondary branches, respectively, were also counted. The numbers of the branches were also counted from 10-12 confocal laser microscopic fields that were randomly chosen per each cover glass on which the neuronal cells were grown and immunostained with MAP2 antibodies. Analysis of the neurite length and the numbers of the branches was performed with LSM Image Examiner ver. 2.80 (Carl Zeiss).

In the immunohistochemical analysis, the number of the cells immnunostained against each antibody was counted from 5 or 6 hippocampal coronal sections. For each staining analysis, every fifth cryosections of the hippocampal region of brain tissues (AP: bregma −4.5 to −4.3 mm) were taken for immunostaining. The number of the immnunostained cells was represented as an average of number of cells in the hippocampal region of one brain slice.

The number of the VGluT1- and GAD-positive cells in the brain slice samples 1 week and 4 weeks after the administration of soyasaponin I was presented per microscopic field (×400-VGluT1, ×800-GAD67) and the number of the ChAT-positive cells was presented per each hippocampal region (10-12 confocal laser microscopic fields). 5-11 animals were used per group. Western blotted membranes were analyzed using the multi-gauge, bio-imaging program on LAS-4000 Mini (Fujifilm Life Science, Stamford, Conn., USA). Densitometeric analysis of the expression ratios of VGluT1/β-actin, GAD65/67/β-actin and ChAT/β-actin was normalized to the vehicle group.

All the behavioral data, cell count data and densitometric data were expressed as mean±standard deviation, and statistical significance was analyzed by one-way analysis of variance (ANOVA) followed by the Newman-Keuls multiple comparison test or unpaired t test (GraphPad Prism, ver. 5.01). Power analysis was conducted using G*Power ver. 3.1.7 (Faul F et al., Behav Res Methods, 2009). Statistical significance was set at p<0.05.

### Test Result 1

### Effect of Soyasaponin I on Promotion of Differentiation and Proliferation of New-Born Neural Precursor Cells in Memory-Deficient Model Rats (Experimental Example 2)

If new-born neural precursor cells generated within 1 week proliferate and get incorporated into the neural network and neuronal cell types are increased as a result thereof, the ability of memory formation can be maintained. The result of the behavioral tests conducted to investigate the effect of soyasaponin I on proliferation of neural precursor cells is shown in FIGS. 4B-4G, and the immunohistochemical analysis result is shown in FIGS. 5A-5E.

(1) Y-Maze Test Result

As can be seen from FIGS. 4B-4C, in the Y-maze test, spontaneous alterations increased significantly in the soyasaponin I-administered group, by 78.57%, compared with the non-soyasaponin I-administered group (IBO) (### p<0.001 by the Newman-Keuls multiple comparison test, the IBO group was compared with the normal group, ** p<0.01 by the Newman-Keuls multiple comparison test, the soyasaponin I 10 mg/kg group was compared with IBO group; F_{2,27}=25.57, one-way ANOVA, p<0.0001), whereas the number of total entries was not significantly different among all the groups of the memory-deficient model rats.

(2) Passive Avoidance Test Result

As can be seen from FIG. 4D, in the passive avoidance test, the latency time of the soyasaponin I-administered group was recovered dramatically as compared with the non-soyasaponin I-administered group (IBO), to 89.65% of that of the normal group) (### p<0.001 by the Newman-Keuls multiple comparison test, the IBO group was compared with the normal group, *** p<0.001 by the Newman-Keuls multiple comparison test, the soyasaponin I 10 mg/kg group was compared with IBO group; F_{2,27}=40.57, one-way ANOVA, p<0.0001).

(3) Morris Water Maze Test Result

As can be seen from FIGS. 4E-4G, in the training trial session of the Morris water maze test, the escape latency time for finding the hidden platform of the normal group and the soyasaponin I-administered memory-deficient group declined progressively during the training period of 4 consecutive days, as compared with the non-soyasaponin I-administered group (IBO) (FIG. 4E). In particular, on the third and fourth days, the escape latency time of the soyasaponin I-administered group was decreased as compared to the ibotenic acid-administered group (day 3, F_{2,29}=14.29, p<0.0001; day 4, F_{2,29}=9.985, p=0.0004).

And, as can be seen from FIG. 4F and FIG. 4G, in the probe trial session, whereas the number of crossings across the target probe and the swimming time in the target quadrant showed reduced tendency for the non-soyasaponin I-administered group (IBO), the soyasaponin I-administered group showed recovery to almost those of the normal group (swimming time, F_{2,29}=21.07, p<0.0001; target crossing, F_{2,29}=25.63, p<0.0001).

These results indicate that the memory improvement in the behavioral tests resulting from the soyasaponin I administration lasted for 4 weeks after the administration of soyasaponin I.

(4) Immunohistochemical Analysis Result

In order to investigate the effect of soyasaponin I on differentiation of newly born neural precursor cells, it was investigated whether BrdU-positive cells express a mature neuron marker and cell subtype markers (NeuN, VGluT1, GAD67) at the same time 4 weeks after the oral administration of soyasaponin I. The result is shown in FIGS. 5A-5E.

The number of BrdU-positive cells in the hippocampal tissue slices at 4 weeks after the oral administration of soyasaponin I (FIG. 5B) was reduced to less than half of that at 1 week after the oral administration of soyasaponin I (FIG. 2B). In contrast, the number of BrdU-positive cells and newly born dentate granular cells (DGCs) was approximately 3.5-fold higher in the soyasaponin I-administered group than in the non-soyasaponin I-administered group (IBO) (normal group n=5, ibotenic acid-administered group n=5, soyasaponin I 10 mg/g group n=5; F_{2,12}=9.975, p=0.0028 by one-way ANOVA). These results indicate that the survival of newly born cells was maintained for at least 4 weeks.

Also, as can be seen from FIG. 5A and FIG. 5C, the number of cells expressing both NeuN (mature neuron marker) and BrdU was 3 times higher for the soyasaponin I-administered group as compared to the ibotenic acid-administered group (normal group n=4, IBO group n=5, soyasaponin I 10 mg/g group n=5; F_{2,11}=11.18, p=0.0022 by one-way ANOVA)

And, as can be seen from FIG. 5A, the number of BrdU-positive cells expressing VGluT1 and GAD67 at the same time was times and 2.8 times higher, respectively, for the soyasaponin I-administered group as compared to the IBO group (normal group n=3, ibotenic acid-administered group n=3, soyasaponin I 10 mg/g group n=3; GAD67+BrdU, F_{2,6}=30.33, p=0.0007 by one-way ANOVA; VGluT1+BrdU, F_{2,6}=10.94, p=0.01 by one-way ANOVA).

However, there was no significant difference in the ratio of the BrdU-positive cells that express VGluT1, NeuN or GAD67 at the same time (FIGS. 5B and 5C).

Among the hippocampal cells expressing an astrocyte marker (GFAP), a dopaminergic neuronal marker (TH) and a cholinergic neuronal marker (ChAT), BrdU-positive cells were barely detected in any group, probably because the new-born cells mostly migrate to the granular cell layer consisting of glutamatergic and GABAergic neurons.

Also, it was found that the cholinergic neurons, which were increased at 1 week after the oral administration of soyasaponin I, were maintained at a similar level until 4 weeks after the oral administration (normal group n=5, ibotenic acid-administered group n=5, soyasaponin I 10 mg/g group n=5; F_{2,12}=15.78, p=0.0004 by one-way ANOVA), as can be seen from FIGS. 5d and 5e, when the total endogenous ChAT-positive cells were immunostained. This result suggests that the administration of soyasaponin I neuroprotects the cholinergic neurons from degeneration induced by ibotenic acid administration in the memory-deficient model.

The neural precursor cells differentiated preferentially into neurons rather than into astrocytes. The neuronal cell types were glutamatergic and GABAergic rather than dopaminergic or cholinergic.

As such, it was confirmed that soyasaponin I has the effect of promoting survival of neurons, proliferation of newly generated neural precursor cells, and differentiation into neurons suitable for the transplantation site.

### Test Result 2

### Effect of Soyasaponin I on Promotion of Differentiation and Proliferation of Embryonic Neural Stem Cells or Neural Precursor Cells (Experimental Example 3)

### (1) Effect of Promoting Proliferation of Embryonic Neural Stem Cells or Neural Precursor Cells

The immunohistochemical analysis result for Ki67 in Experimental Example 3. (2) is shown in FIG. 6A and FIG. 6B. Ki67 is a cell proliferation marker. The test group treated with 2 μM soyasaponin I (Soya-I) showed 2.5-fold increase in the number of proliferating cells as compared with the normal group (Vehicle), and the proliferation of the neural precursor cells increased rapidly in a soyasaponin I concentration-dependent manner (normal group n=3, soyasaponin I 0.5 μM group n=3, 1 μM group n=3, 2 μM group n=3; F_{3,8}=9.426, p=0.0053 by one-way ANOVA).

Accordingly, it was confirmed that soyasaponin I has an excellent effect of promoting proliferation of neural stem cells or neural precursor cells. The proliferation of neural stem cells is a different procedure from their differentiation. Although many researchers have attempted large-scale culture of stem cells, human adult neural stem cells are particularly difficult to culture in vitro and have limited ability to proliferate. For this reason, studies on the human adult neural stem cells are at a standstill. The inventors of the present invention have first confirmed that the composition containing soyasaponin I according to the present invention has a remarkable effect of promoting proliferation of neural stem cells or neural precursor cells.

### (2) Effect of Promoting Differentiation of Embryonic Neural Stem Cells or Neural Precursor Cells

After treating the neural precursor cells with soyasaponin I according to Experimental Example 3. (3) and culturing for 6 days for differentiation, immunostaining was performed using neuronal differentiation markers (NeuN, TUJ1) and a neurite outgrowth marker (MAP2). The result is shown in FIGS. 6C-6H.

FIG. 6C and FIG. 6D show a result of immunostaining using the neuronal differentiation marker NeuN (normal group n=12, soyasaponin I 0.5 μM group n=12, soyasaponin I 1 μM group n=14; F_{2,35}=14.21, p<0.0001 by one-way ANOVA). Soyasaponin I promoted differentiation into mature neurons in a concentration-dependent manner and, as a result, the number of NeuN-expressing cells was increased.

FIG. 6E and FIG. 6F show a result of immunostaining using the neuronal differentiation marker TUJ1 (normal group n=8, soyasaponin I 0.5 μM group n=8; p<0.0001 by unpaired t test). The treatment with soyasaponin I resulted in about 4-fold increase in TUJ1-expressing cells as compared to the normal group. The treatment of the neural precursor cells with soyasaponin I promoted differentiation into late-stage neural precursor cells.

FIG. 6G and FIG. 6H show a result of immunostaining using the neurite outgrowth marker MAP2 (MAP2-positive cells; normal group n=5, soyasaponin I 0.5 μM group n=5; p=0.0073 by unpaired t test). The treatment of the hippocampal neural precursor cells with 1 μM soyasaponin I for 4 days increased neurite length 2.2-fold as compared to the normal group. Accordingly, it was confirmed that soyasaponin I has an effect of promoting neurite outgrowth.

In the hippocampal neural precursor cell cultures, astrocytes expressing glial fibrillary acidic protein (GFAP) were hardly observed. GFAP is an intermediate filament protein and constitutes glial filaments.

Also, as can be seen from FIG. 6G, the average neurite length and the numbers of both primary and secondary branches were markedly increased in the soyasaponin I-treated group (normal group n=17, soyasaponin I 1 μM group n=17; p<0.0001 by unpaired t test).

Accordingly, it was confirmed that soyasaponin I is effective in promoting differentiation, in addition to proliferation, of neural stem cells or neural precursor cells.

The result of immunoblotting of neural precursor cells according to Experimental Example 3. (4) is shown in FIGS. 7A-7C. Vesicular glutamate transporter 1 (VGluT1) is a glutamatergic neuronal marker, GAD67 is a GABAergic neuronal marker, and choline acetyltransferase (ChAT) is a cholinergic neuronal marker.

As can be seen from FIG. 7A, the expression level of ChAT protein was increased 1.9-fold, respectively, in the test groups as compared to the normal group (normal group n=4, soyasaponin I 0.5 μM group n=4, soyasaponin I 1 μM group n=4; soyasaponin I 2 μM group n=4; F_{3,12}=4.584, p=0.0232 by one-way ANOVA). However, the expression of VGluT1 or GAD65/67 proteins increased relatively little after the treatment with soyasaponin I for 6 days (FIG. 3B and FIG. 3C).

As such, it was confirmed that soyasaponin I promotes proliferation and differentiation of hippocampal neural precursor cells primarily cultured from the embryo.

### Test Result 3

### Effect of Soyasaponin I on Promotion of Neuronal Regeneration in Memory-Deficient Model Rats (Experimental Example 1)

### (1) Y-Maze Test Result

FIG. 1B and FIG. 1C show the Y-maze test result of Experimental Example 1. (1) for evaluating spatial memory. The rats faced a choice in selecting a pathway in the Y-shaped track. Spontaneous alterations in arm entries were lower in the groups 1 and 2 as compared to the group 3 (Sham) (p<0.001 by the Newman-Keuls multiple comparison test). However, the decreased memory observed in the group 2 (IBO) was improved in the soyasaponin I-administered group 1 (Soya-I) (p<0.01 by the Newman-Keuls multiple comparison test; F_{4,34}=10.64, p<0.0001 by one-way ANOVA).

Through this, it was confirmed that soyasaponin I has an effect of recovering and improving impaired learning and memory abilities in the memory-deficient model by promoting proliferation of newly born neural precursor cells. Accordingly, it was confirmed that the composition containing soyasaponin I according to the present invention can be used as a pharmaceutical composition for preventing or treating various neurological diseases such as Alzheimer's disease, Parkinson's disease, epilepsy, depression, cerebral ischemia, etc., which are associated with impaired learning and memory.

### (2) Passive Avoidance Test Result

FIG. 1D shows the passive avoidance test result of Experimental Example 1. (2). 24 hours after the rats received a shock, the latency time for the rats to enter the dark room was measured. The group 2, i.e., the memory-deficient model, showed markedly decreased latency time as compared to the normal group 3 (Sham). In contrast, the latency time of the group 1, which is the soyasaponin I-administered memory-deficient group, showed improvement in latency time.

Through this, it was confirmed that soyasaponin I has an effect of recovering impaired learning and memory abilities and enhancing memory in the memory-deficient model by promoting proliferation of newly born neural precursor cells. Accordingly, it was confirmed that the composition containing soyasaponin I according to the present invention can be used as a pharmaceutical composition for preventing or treating various neurological diseases such as Alzheimer's disease, Parkinson's disease, epilepsy, depression, cerebral ischemia, etc., which are associated with impaired learning and memory.

### (3) Effect of Soyasaponin I on Promotion of Proliferation of Differentiated Neurons

The immunohistochemical analysis result for BrdU in Experimental Example 1. (4) is shown in FIG. 2A and FIG. 2B. To examine whether soyasaponin I promotes proliferation and differentiation of hippocampal cells, which are responsible for the formation of learning and memory, the rat brain tissues were immunostained with a cell proliferation marker (BrdU). As can be seen from FIG. 2B, the number of endogenous BrdU-positive neural precursor cells in the hippocampal region was increased by the oral administration of soyasaponin I (Soya-I) as compared to the non-administered memory-deficient model (IBO).

In particular, as can be seen from FIG. 2B, the number of the BrdU-positive cells in the dentate gyms (DG) of the hippocampus increased significantly for all doses (5, 10, 20 mg/kg) of orally administered soyasaponin I. The largest increase in the number of BrdU-positive cells was observed when 10 mg/kg soyasaponin I was administered (normal group n=4, ibotenic acid-administered group n=3, 5 mg/kg soyasaponin I-administered group n=3, 10 mg/kg soyasaponin I-administered group p n=3, 20 mg/kg soyasaponin I-administered group n=3; F_{4,11}=18.18, p<0.0001 by one-way ANOVA).

Accordingly, it was confirmed that soyasaponin I has an effect of remarkably promoting proliferation of hippocampal neurons in memory-deficient rats and that soyasaponin I can prevent, treat and improve neurological diseases associated with learning and memory abilities.

FIGS. 3A-3E show the result of the immunohistochemical analysis in Experimental Example 1. (5) for investigating the effect of promoting proliferation and differentiation of neurons in the hippocampus and the entorhinal cortex, which are involved in learning and memory. The result of immunohistochemical staining of the neurons in the hippocampus and the entorhinal cortex using neuronal subtype differentiation markers is shown. The markers were GAD67 (GABAergic neurons), VGluT1 (glutamatergic neurons), ChAT (cholinergic neurons) and OX42 (reactive microglia).

When compared with the normal group (Sham), the numbers of GAD67-, VGluT1- and ChAT-positive cells decreased markedly in the hippocampus for the non-soyasaponin I-administered memory-deficient group (IBO).

TABLE 1
(unit: %)

| | IBO | Sham |
|---|---|---|
| GAD67 | 43.02 ± 3.72 | 100 ± 11.43 |
| VGluT1 | 72.44 ± 3.86 | 100 ± 6.50 |
| ChAT | 52.07 ± 7.38 | 100 ± 1.64 |

This result indicates that injection of ibotenic acid into the entorhinal cortex, where many neurons project toward the hippocampus, decreases the numbers of the major neuronal cell types that participate in the formation of hippocampal memory, such as glutamatergic, GABAergic and cholinergic neurons.

When soyasaponin I was injected to the memory-deficient model, the expression of the markers was markedly increased as compared when it was not injected (IBO).

The expression level of the GABAergic neuronal marker GAD67 was compared as follows. At the three doses tested (5, 10, 20 mg/kg), the soyasaponin I-administered group 1 showed approximately 1.66- to 1.76-fold (6.83±1.47 to 7.86±1.62 cells per hippocampal slice) increase in the number of GAD67-positive cells in the hippocampus as compared to the IBO group (3.81±0.63 cells, normal group n=5, ibotenic acid group n=5, 5 mg/kg soyasaponin I group n=5, 10 mg/kg soyasaponin I group n=5, 20 mg/kg soyasaponin I group n=5; F_{4,20}=5.191, p=0.0049 by one-way ANOVA).

The expression level of the glutamatergic neuronal marker VGluT1 was compared as follows. The number of VGluT1-positive cells increased up to the level of the normal group (57.33±2.43 cells per microscopic field at 10 mg/kg) as compared to the IBO group (normal group n=5, ibotenic acid group n=5, 5 mg/kg soyasaponin I group n=5, 10 mg/kg soyasaponin I group n=5, 20 mg/kg soyasaponin I group n=5; F_{4,20}=8.127, p=0.0005 by one-way ANOVA).

The expression level of the cholinergic neuronal marker ChAT was compared as follows. The number of ChAT-positive cells in the hippocampus increased 1.39- to 2.23-fold (17.64±4.23 cells to 20.93±4.50 cells) for the group 1 as compared to the group 2 (7.30±1.32 cells; normal group n=5, ibotenic acid group n=5, 5 mg/kg soyasaponin I group n=5, 10 mg/kg soyasaponin I group n=5, 20 mg/kg soyasaponin I group n=5; F_{4,20}=3.314, p=0.0308 by one-way ANOVA).

Through this, it was confirmed that soyasaponin I promotes proliferation of neural precursor cells and differentiated neurons such as GABAergic, glutamatergic and cholinergic neurons and, thereby, facilitates neuronal regeneration. Accordingly, the composition containing soyasaponin I according to the present invention can be used as a composition for promoting proliferation of neural stem cells or neural precursor cells.

For OX42 as the marker for reactive microglia, when the intensity of OX42-positive cells in the DG and CA1 regions of the non-soyasaponin I-treated group (IBO) was normalized to the intensity of the normal group, the normalized intensity of the ibotenic acid group increased significantly as compared to the normal group, as can be seen from FIG. 3A. In contrast, the normalized intensity of the soyasaponin I-treated group decreased in a dose-dependent manner in both the DG and CA1 regions.

FIG. 3E shows the expression intensity of OX42 in the memory-deficient model as compared to the normal model (Sham) (normal group n=5, ibotenic acid group n=5, 5 mg/kg soyasaponin I group n=5, 10 mg/kg soyasaponin I group n=6, 20 mg/kg soyasaponin I group n=5; CA1, F_{4,21}=12.83, p<0.0001 by one-way ANOVA; DG, F_{4,21}=3.559, p=0.0229 by one-way ANOVA). OX42 expression was increased when the normal model was treated with ibotenic acid to induce memory impairment. However, the OX42 expression was decreased again to the level of the normal model when treated with soyasaponin I (Soya-I).

Taken together, these results suggest that soyasaponin I has various effects of affecting neuronal regeneration and protection by inhibiting degeneration and inflammation in the memory-deficient model induced by ibotenic acid. Accordingly, it was confirmed that the soyasaponin I according to the present invention has the effect of promoting proliferation of neural stem cells or neural precursor cells and, thereby, providing neuronal regeneration and protection.

As described, the composition containing soyasaponin I according to the present invention has the effect of promoting differentiation or proliferation of neural stem cells or neural precursor cells and recovering damaged neurons. Accordingly, the composition containing soyasaponin I according to the present invention may be effectively used for large-scale culturing of stem cells for laboratory use or transplantation and for preventing or treating neurological diseases by regenerating the nervous systems.

While the exemplary embodiments have been shown and described, it will be understood by those skilled in the art that various changes in form and details may be made thereto without departing from the spirit and scope of the present invention as defined by the appended claims. In addition, many modifications can be made to adapt a particular situation or material to the teachings of the present invention without departing from the essential scope thereof. Therefore, it is intended that the present invention not be limited to the particular exemplary embodiments disclosed as the best mode contemplated for carrying out the present invention, but that the present invention will include all embodiments falling within the scope of the appended claims.

1. A method for promoting differentiation or proliferation of neural stem cells or neural precursor cells by administration of a composition that comprises soyasaponin I represented by Chemical Formula 1:

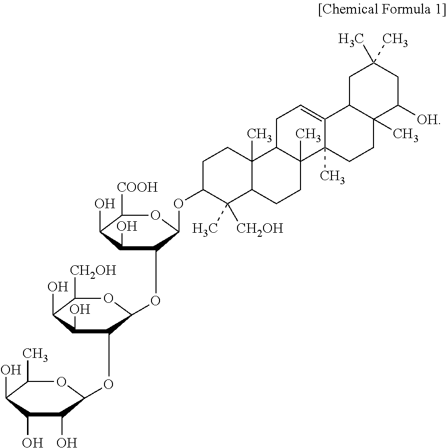

2. The method according to claim 1, wherein the neural stem cells or neural precursor cells are isolated cells.

3. The method according to claim 1, wherein the soyasaponin I is extracted from soybean.

4. The method according to claim 3, wherein the soybean is one of bean, germinated soybean or soybean sprout.

5. The method according to claim 1, wherein the composition further comprises an inducing agent which induces proliferation or differentiation of neural stem cells or neural precursor cells.

6. The method according to claim 5, wherein the inducing agent comprises FGF, EGF or retinoic acid.

7. (canceled)

8. A method for preventing or treating neurological diseases by promoting differentiation or proliferation of neural stem cells or neural precursor cells by administration of a composition that comprises soyasaponin I represented by Chemical Formula 1:

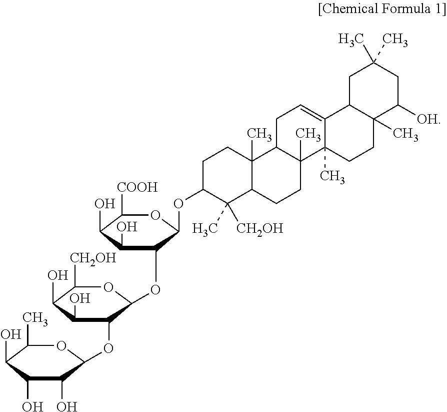

9. The method according to claim 8, wherein the neurological disease is selected from the group consisting of dementia, Parkinson's disease, Alzheimer's disease, Pick's disease, Huntington's disease, epilepsy, stroke, ischemic brain disease, degenerative brain disease, memory impairment, traumatic central nervous system disease, spinal cord injury, peripheral nerve injury, amyotrophic lateral sclerosis, peripheral nerve disease, behavioral disorder, developmental disorder, mental retardation, Down's syndrome, and schizophrenia.

10. (canceled)
